(19) United States
(12) Patent Application Publication
(10) Pub. No.: US 2009/0124013 A1
(43) Pub. Date: May 14, 2009

(54) Stem-Like Cells

(76) Inventor: George S. Kouns, Jasper, AL (US)

Correspondence Address: KNOBLE, YOSHIDA & DUNLEAVY, EIGHT PENN CENTER, SUITE 1350, 1628 JOHN F KENNEDY BLVD, PHILADELPHIA, PA, 19103, US

(21) Appl. No.: 11/815,167

(22) PCT Filed: Aug. 15, 2005
(86) PCT No.: PCT/US05/28900

§ 371 Date: Jul. 31, 2007

Related U.S. Application Data
(60) Provisional application No. 60/537,746, filed on Aug. 16, 2004.

(51) Int. Cl. C12N 15/87; C12N 5/08

(52) U.S. Cl. 435/461; 435/366

(57) ABSTRACT

A method for the production and use of multipotential stem-like cells is disclosed. The preparation utilized in this method is characterized by the contact of low level electrical currents with cultures of fibroblasts or other -blast cells enriched by fibroblast growth factor and other nutrients. The electrical current is conducted by means of silver electrode(s) brought into contact with the fibroblast preparation or other -blast cell preparation cultured for that purpose. The cells of the preparation may be used in applications that require the use of stem cells, including therapeutic applications, without the need for human fetuses or human umbilical cords or penetrating human bones to extract bone marrow. The cells thus produced have the ability to redifferentiate into endoderm, ectoderm and mesoderm to form any tissue of the body except the lens of the eye. Any cell found in the blood may be copied and multiplied. Any tissue of the body may be copied and multiplied with the lone exception of the lens of the eye as noted above.

### CROSS REFERENCES TO RELATED APPLICATIONS

### Priority

The present application claims priority to U.S. Provisional Application No. 60/537,746 filed by Aug. 16, 2004 by George Samuel Kouns.

### FEDERALLY SPONSORED RESEARCH OR DEVELOPMENT

Not Applicable

### BACKGROUND OF THE INVENTION

In a fundamental explanation of stem cells, they may be thought of as being able to become whatever kind of cell they touch by absorbing some of the messenger RNA from a neighboring cell and then using it as a blueprint to make a new copy of that neighboring cell. Even if that neighboring cell has been injured, damaged or depleted, the messenger RNA will still code for the same kind of cell that was present before the injury, damage, or depletion occurred. In this fashion, stem cells, or cells that will perform the same function as stem cells, are capable of growing into any organ and/or any type of tissue. The term “stem-like cells” herein refer to cells which are essentially functionally equivalent to stem cells.

In a study performed by Evans, et al., Nature 292:145-159, 1981, the embryonic stem cells of mice were shown to be capable of being derived in vitro. In a later study, Williams, et al., in the November 1992 edition of Nature 336:684-692 showed that the same type of cell could be maintained in an undifferentiated state for an extended period by the use of leukemia inhibitory factor. These studies showed that mouse embryonic stem cells will readily differentiate into many types of tissue. This provides indication that stem cells and stem-like cells of other animals such as humans, can conceivably behave in a similar fashion.

Further, Time-Life Books reported saturating surgical sponges with fibroblast growth factor and placing them next to liver tissue in lab animals. The reported result was a small liver “organoid”. To maintain the new hepatic tissue, a constant supply of fresh fibroblast growth factor was required. At that time, this requirement was cost prohibitive. Also, it is generally known that fibroblast growth factor can be induced by heat and other stresses. The option to stress fibroblast with low level electric currents similar to what is found in the human body in the so-called “current of injury” at an injury site was considered. The objective of a low-level electric current stress was to stimulate the release of fibroblast growth factor. The work of Robert O. Becker, “The Body Electric”, published in 1985 was instructive. Becker described the use of silver electrodes to deliver low level currents to fibroblasts in a Petri dish. He reported that the fibroblasts became multipotential dedifferentiated cells after stimulation with electrically charged silver in the treatment of non-union fractures and osteomyelitis. Dr. Becker's objective focused on facilitating the healing of fractures and infections.

### REFERENCES CITED

### US Patent Documents

| | | | |
|---|---|---|---|
| 5,166,065 | November, 1992 | Williamson, et. al. | 435/325 |
| 5,340,740 | August, 1994 | Pettite, et. al. | 435/325 |
| 5,449,620 | September, 1995 | Khillian | 435/325 |
| 5,453,357 | September, 1995 | Hogan | 435/7 |
| 5,591,625 | January, 1997 | Gerson, et. al. | 435/325 |

### Foreign Patent Documents

WO 94/03585

### OTHER REFERENCES

Becker, R. O. The Body Electric, William Morrow and Company, New York, N.Y., 1985
Bongso, A., et. al., “Isolation and culture of inner cell mass cells from human blastocyats” Human Reproduction, 9(1):2100-2117 (1994)
Doctschman, T., et. al., Establishment of hamster blastocyst derived embryonic stem cells, Developmental Biology 127:224-227 (1998)
Evans, M., et. al., Establishment in culture of pluripotential cells from mouse embryos, Nature, 292:154-156 (1981)
Evans, M., et. al., Derivation and preliminary characterization of pluripotent cell lines from porcine and bovine blastocysts, Theriogenology 33(1):125-128 (1990)
Nichols, et. al., Establishment of germ-line-competent embryonic stem cells using differentiation inhibiting activity, Development, vol. 110, pp. 1341-1348, 1990.
Notarianni, B., et. al., “Maintenance and differentiation in culture of pluripotential embryonic cell lines from pig blastocysts,” Journal of Reprod. Fert. Suppl., 41:51-56 (1990)
Notarianni, E., et. al., “Derivation of pluripotent embryonic cell lines from the pig and sheep,” J. Rep. And Fert. 43 225-260 (1991)
Pledrahita, J. Discussion on “Studies on the isolation of embryonic stem cells: Comparative behavior of murine, porcine and ovine species,” University of California Davis, 1989.
Strojek, R., et. al., “A method for cultivating morphologically undifferentiated embryonic stem cells from porcine blastocysts,” Theriogenology, 33 901-913 (1990)
Thompson, John (see in Time-Life Books: ISBN D-7835-1048-9 Library of Congress)
Williams, R., et. al., “Myeloid leukaemia inhibitor factor maintains the developmental potential of embryonic stem cells,” Nature 336:684-692 (1988)

### BRIEF SUMMARY OF THE INVENTION

The objective of this invention teaches the extension of the work of Thomson and Becker and provides a mechanism and method to make these multipotential dedifferentiated stem-like cells available on a large scale to perform the same function as embryonic stem cells and stem cells derived from bone marrow. In a present preferred embodiment of this invention the donor cells needed to initiate this process of producing stem-like cells may be obtained from the skin. Fibroblast cells from a skin sample are a preferred source of the donor cells.

The foregoing and other and further objects, features, and advantages of the Invention will be indicated in the appended Claims or will be apparent upon an understanding of the following more particular description of preferred embodiments as illustrated in the accompanying drawings in which a reference character refers to the same part throughout the various views as set forth in the two (2) Indices immediately following the list of Figures immediately below. The drawings are not necessarily to scale, emphasis instead being placed upon illustrating the principles of the Invention. Moreover, various objects, features, and advantages of the Invention not referred to herein will occur to one skilled in the art upon employment of the Invention in practice.

It is accordingly an object of the present invention to provide a methodology and rational for the production of non-embryonic stem-like cells in vitro which are suitable for many of the uses requiring embryonic stem cells, including but not limited to use as a therapeutic agent in the treatment of some diseases and some forms of tissue injuries. The methodology involves the dedifferentiation of fibroblasts, in vitro, by means of stimulation using low voltage and low amperage electrical current delivered through silver electrodes to a collection of donor cells which had been placed in a Petri dish. Silver ions thus released act as a stress catalysts in the dedifferentiation process.

In this methodology, the electric current is applied for 120 hours (5 days), at which time sample specimens from the north, south, east and west points of the periphery of the collection as well as a specimen from the center are examined under a microscope to identify them histologically as being either stem-like cells or fibroblasts. When all five samples contain all stem-like cells, it its inferred that the remaining cells in the Petri dish have also completed dedifferentiation. If all the fibroblasts in the sample had not changed to stem-like cells, then they would either remain fibroblasts or form scar tissue which is of no clinical significance.

At such time, it would be acceptable to use a mixture of dedifferentiated stem-like cells with dedifferentiated mesoderm cells and then redifferentiate them into muscle, bone, cartilage, blood, pancreas, liver, kidney, lung, etc.

In addition to using the skin to obtain fibroblast donor cells, other sources of donor cells include ectodermal, entodermal, and mesodermal cells, which may be easily and safely biopsied from a donor patient. Cells from any of these sources can be dedifferentiated into stem-like cells by the continued application of silver ions driven by electrical current. Fibroblast cells are preferred over other “-blast” cells because they can be harvested from the skin on an outpatient basis under local anesthetic.

As one example of use of the stem-like cells produced in accordance with this invention, if the stem-like cells produced in this manner are injected into the donor patient's liver, they will redifferentiate into endoderm cells and then mature into hepatocytes (liver cells) just as stem cells behave when forming liver cells a fetus. This example of the application of this invention is the injection of the stem-like cells into an impaired organ of the patient who donated the fibroblasts (or other “-blast” cells, provided they are not subject to any toxicities or infections. New host cells will grow from the stem-like cells as the stem-like cells absorb the host organ's messenger RNA. Stem-like cells derived from fibroblast (or other “-blast” cells) of any tissue-compatible donor would work as well.

As a second preferred embodiment of this invention, organs of all various tissue types may be grown in the lab and stored in “organ banks” like blood is stored, refrigerated in blood banks. In like fashion kidney transplantation would become an option for the millions of patients around the world on dialysis. All such organs can be grown and stored for shipment and transplantation on request. Thus far, no one has cultured a brain or heart.

This invention should allow growth of a heart from stem-like cells once the technique is developed to introduce neuroblasts at the midline septum forums to facilitate the foundation of the nerve bundle that keeps the heart beating.

Spinal cord repair has been done on humans successfully in Korea as recently reported in the literature. The brain may be described in laymen's terms as a “wadded-up” extension of the spinal cord. Again, this invention will allow growth of brain tissue such that once technology is developed to enable the implant or installation of such stem-like cell developed brain tissue into a brain damaged patient.

In summary regarding these examples, stem-like cells do not have to be converted into any target tissue ex-vivo to be used as a therapy. They are just injected “as-is” into the damaged organ and they form new, healthy organ cells. In addition, it is possible to “seed” a stem-like cell population with a liver cell and grow a new liver, for example, like a mature grain stalk grows from a single kernel of corn. Any other organ may likewise be grown and used in therapy except those like the heart and brain where implementing technology has yet to be developed as illustrated in above.

According to one feature of the invention, the above and other objectives are carried out according the present invention by a Petri style dish utilized for the ex vivo growth and maintenance of fibroblasts and the dedifferentiation and redifferentiation of said fibroblasts ex vivo.

According to another feature of the invention, the silver electrodes may be disinfected and reused for new cultures of fibroblasts whether they are differentiated or redifferentiated.

According to features of the invention, a container, for example, a Petri style dish, may be 1) reused and 2) used for storage of materials processed. In another feature, the Petri style dish may be used to culture specific types of tissues and/or cells by bringing the dedifferentiated fibroblasts in contact with cells of the desired tissue classification and subjecting the combination of cells to low voltage, low amperage electrical stimulation to redifferentiate them into a new cell type. The device is capable of holding growth factors, inhibitory factors and/or other nutrients, enzymes or other materials that will catalyze and facilitate the dedifferentiation and/or redifferentiation of fibroblasts into multipotential dedifferentiated cells and thereafter into cell types and tissue classifications that could be used as therapeutic agents for select populations of patients.

The process of this invention involves obtaining, from a patient, seed cells representing the cell types to be used in the repair and or replacement or cure of the diseased organ of the patient. Because the donor cells are from the patient and the resulting cells and tissue used in the treatment of the patient, there is no necessity for tissue typing, immunosuppression, or waiting for a donor. An advantage of this invention is that there is little chance of rejection since the patient is receiving his or her own DNA. In some scenarios this aspect of this invention represents a life saving time acceleration of effective treatment of many diseases or injuries.

Another advantage of this invention is that it allows production of a large number of stem-like cells and, subsequently, replicated target seed cells in vitro to be therapeutically sufficient for the donor patient.

Another advantage of this invention is the option to select donor skin (fibroblast) cells from a patient's parent, sibling, children, or other person where compatibility of cell type has been pre-determined (e.g. tissue typing).

In addition, another advantage of this invention is that production of stem-like cells does not require employing the use of human embryos or human umbilical cords. Such employment is objected to by some persons, religions, cultures, political parties, or governments or the like. In some scenarios, availability of stem-like cells using the methods and/or apparatus or arrangement of this invention may be the only source of stem-like cells available to much of the human population or the scientific community.

In general terms, the present invention teaches the culture and use of stem-like cells without use of aborted fetuses, umbilical cord blood products or bone matter. Specifically, the present invention is the dedifferentiation of fibroblasts into multipotential dedifferentiated cells to be used as treatments and therapies for diseases and scientific investigations that would respond favorably to stem cell theory. It teaches a method and apparatus for the production and use of stem-like multipotential cells by dedifferentiation of -blast cells, such as fibroblast, osteoblast, erythroblasts, and neuroblast cells.

While the above features have been found to be useful, the embodiments of this current invention are not limited to the specific embodiments identified above. Other containers and apparatus, other than Petri style dishes, may be used in bioprocessing steps to accomplish the intended functions illustrated with the Petri style dish. “Stress catalysts” other than electrical generated stress catalysts may also be employed. Also, electrodes and ions and catalysts, other than silver such as titanium or stainless steel, may be useful in accomplishing this invention. Similarly, various time variations of voltage and current may be used to produce the stress applied to the fibroblast cells described in the above process. Similarly, a pulsed electromagnetic field may be useful to induce the described stress on the fibroblast cells. Also, other embodiments may alter the sizes, spacings, and orientations of the containers and electrical apparatus described above. In addition, the embodiments described above use examples of human cells and disease and injury treatment. The invention's methods, apparatus, arrangements, and compositions developed can involve animal cells as well as human cells and can involve development of tissue and organs for animals as well as human tissue and organs and can be used in research to develop enhanced human or animal tissue or organs employing “spliced” genes in the seed cells employed.

And while the Invention has been particularly shown and described with reference to the preferred embodiments thereof, it will be understood by those skilled in the art, that various alterations in form and detail may be made therein without departing from the spirit and scope on the Invention.

### BRIEF DESCRIPTION OF THE DRAWINGS

The essential features and attendant advantages of the present invention will be more fully comprehended when the following written descriptions are considered in connection with the accompanying drawings.

FIG. 1 is a schematic illustration showing the major components of the stem-like cell dedifferentiation production system according to a preferred embodiment of the invention.

FIG. 2 is a schematic illustration of the overall system to convert dedifferentiated multipotential fibroblasts to redifferentiated new cells and tissue and organs according to a preferred embodiment of the invention.

### INDEX OF PART NUMBERS

| | |
|---|---|
| 100 | container dish with side walls |
| 200 | battery |
| 300 | electrical wave generator |
| 400 | silver electrode grid |
| 500 | electrical conducting wire |

### Index of Part Names:

| | |
|---|---|
| 200 | Battery |
| 100 | Container dish with side walls |
| 300 | Electrical wave generator |
| 400 | Electrode grid, silver |
| 500 | Wire, electrical conducting |

### DETAILED DESCRIPTION OF PREFERRED EMBODIMENTS

The present invention provides the technology for achieving a system for production of a large number of stem-like cells using the ex vivo growth and expansion of a small number of fibroblast cells followed by the dedifferentiation of the large number of fibroblast and their conversion to other cell types and forms of tissue. This methodology and system is designed to produce stem-like cells in sufficient quantity to provide therapy and treatment to victims of for example spinal cord trauma and degenerative diseases. It also is designed to culture blood cells to be infused into patients for example who have temporarily lost their ability to maintain said blood cells.

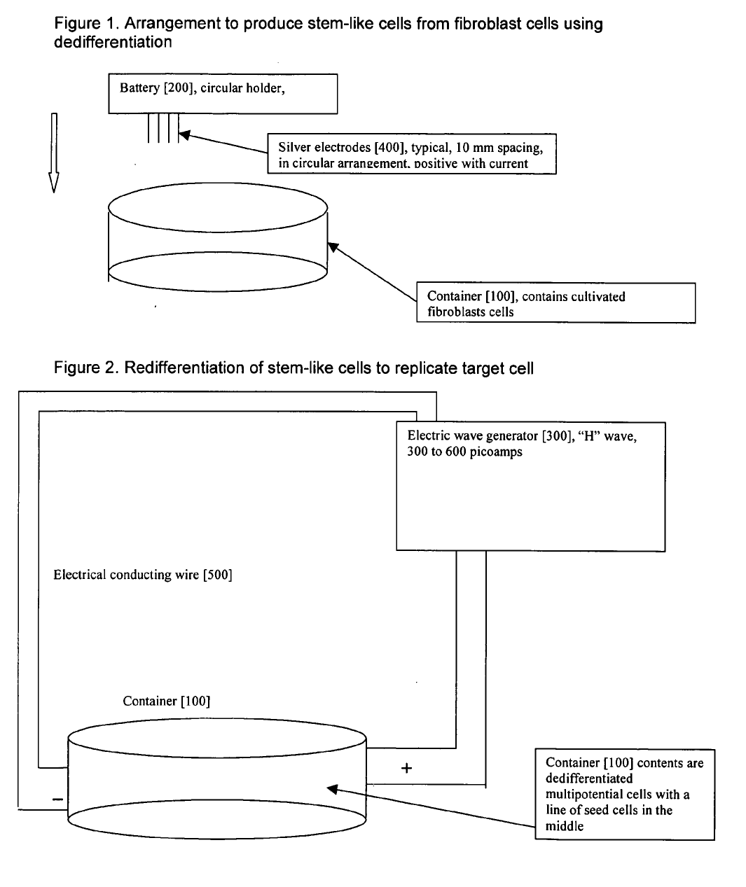

With reference to FIG. 1, the major components of a preferred embodiment of this stem-like cell production system include a washable and reusable glass dish serving as a container (100) with raised sides. This container is designed to hold the cells to be dedifferentiated and/or redifferentiated. Typically, approximately 20 fibroblast cells are cultured from approximately one square inch of an appropriate layer of skin from the patient to be treated with the product cells of this invention. This small number of fibroblast cells is first placed into the container (100). This group of cells is cultured by well-known biological means to increase their number significantly. This step is where growth and expansion of cell numbers occurs. The container (100) may also be the site of final harvest of the finished stem-like cells. As an alternative, cells may be removed from this container (100) and deposited in a different container for incubation, growth and expansion.

Again with reference to FIG. 1, a battery (200) provides electrical power to a grid of silver electrodes (400) each ten millimeters apart and arranged in a generally parallel configuration resembling the bristles of a round brush that fits inside the inner diameter of the container (100) containing the fibroblasts and/or stem-like cells to be processed. A current conducted to the silver electrodes (400) by means of conducting wire (500) of 300 to 600 picoamps is passed through the silver electrodes and into the cell culture accomplishing dedifferentiation of the fibroblasts into stem-like cells capable of redifferentiation into whichever cell type is introduced into the culture of stem-like cells. One biological mechanism theory for this process involves release of “derepressors” that were folded into the membrane of the fibroblast cells. These “derepressors” act depressing repressed genes thus allowing dedifferentiation into a stem-like cell. This step accomplishes converting the fibroblast cells into multipotential dedifferentiated cells (which will serve the same function as stem cells). These stem-like cells may be injected into a patient, for example, into a damaged or diseased organ, for therapeutic treatment.

With reference to FIG. 2, the stem-like cells thus derived from the dedifferentaion of fibroblasts, are induced to redifferentiate into a new cell type by 1) seeding cells of the cell type of which a culture is desired into the matrix of dedifferentiated fibroblasts and 2) administering a current of 300 to 600 picoamps. This current is applied with an “H” wave generator (300) having an amplitude of current of from less than 200 picoamps to one nanoamp or greater, and operating with a waveform frequency of generally from 1 to 10 Hz for one week or longer (depending on the tissue being cultivated) to “actuate” cell membranes and facilitate or prompt release of messenger RNA from the seed cell(s).

At the beginning of this step, an array of “seed cells” from the patient are arranged in the middle of the culture container (100) also containing a number of dedifferentiated multipotential cells previously developed. The current flows through the cell mix and prompts the release of depressors from the membrane(s) of the seed cell(s) into the cellular cytoplasm of the seed cell(s). These derepress genes in the seed cell nucleus allow it to manufacture new messenger RNA from the seed cells. This seed cell messenger RNA instructs the stem-like multipotential dedifferentiated cells to differentiate into replicas of the seed cell.

The process is continued until the entire mix redifferentiates into cells like the seed cell. In this way, cells, tissue and even entire organs may be cultured from donor cells. Likewise, cells tissues and even entire organs may be cultured from donor cells taken from the diseased or injured patient who needs transplantation or needs increased levels of specific types of cells like T-cells, marrow, or organs like thymus, spleen, liver, pancreas, kidney, eye, etc. The replicated donor seed cells may be grown into a large number of replicated donor seed cells, into tissue made of the replicated donor seed cells, or into an organ made from the replicated seed cells and subsequently implanted into the donor patient or suitable recipient.

What is claimed is:

1. An arrangement suitable for the production of multipotential stem-like cells derived from dedifferentiated cells which stem-like cells maintain the potential to redifferentiate to derivatives of ectoderm, endoderm and mesoderm cells said arrangement comprising a container, an array of electrodes, an electrical wave generator, and a composition of cells and medium.

2. An arrangement according to claim 1 wherein the electrical wave generator, is a battery.

3. An arrangement according to claim 2 wherein the battery can produce a current of 300 to 600 picoamps for a period between 1 hour to 1 month.

4. An arrangement according to claim 1 wherein the container is a Petri style dish.

5. An arrangement according to claim 1 wherein the electrical wave generator can produce an electric current profile with a peak current amplitude range of 300 to 600 picoamps at minus 30 millivolts.

6. An arrangement according to claim 5 wherein the electrical wave generator can produce an electric current with a peak current of at least picoamps and from one to 10 hertz for at least one week.

7. An arrangement according to claim 1 wherein the electrodes are silver and arranged essentially parallel, spaced apart 10 mm, extending into the medium and electrically connected to the positive terminal of said source of electricity.

8. An arrangement according to claim 1 wherein the composition of cells and medium contains cells which are fibroblast cells.

9. An arrangement according to claim 1 wherein the composition of cells and medium contains cells which are duplicates of seed cells.

10. An arrangement according to claim 1 wherein the composition of cells and medium contains a mixture of cells which include seed cells, duplicates of seed cells, and fibroblast cells.

11. An arrangement according to claim 1 wherein the derivatives of ectoderm, endoderm and mesoderm cells maintain the potential to grow into tissue.

12. An arrangement according to claim 1 wherein the derivatives of ectoderm, endoderm and mesoderm cells maintain the potential to grow into an organ.

13. A method for the production of multipotential stem-like cells ultimately for use as stem cells such as for the treatment of a patient from which said stem-like cells may be derived from the patient's donated ectodermal, entodermal or mesodermal cells, as harvested from the donor patient, which donated cells are stimulated to dedifferentiate into stem-like cells.

14. The method of claim 13 wherein the preparation of multipotential stem-like cells is derived from dedifferentiated fibroblast cells, which stem-like cells maintain the potential to redifferentiate to derivatives of ectoderm, endoderm and mesoderm cells and tissue, comprising the steps of:
a) harvesting fibroblast cells from the skin of a patient donor;
b) increasing the number of said fibroblast cells in a culture through incubation, growth, and expansion in an environment plus or minus one degree Fahrenheit of body temperature and in an oxygen and nutrient rich environment uniformly perfused with nutrients and oxygen;
c) redifferentiation of the larger number of fibroblast cells by an electrical current emanating from silver electrodes resulting in stem-like multipotential dedifferentiated cells which are dedifferentiated;
d) injecting into an impaired organ of the patient who donated the fibroblast cells, a therapeutic portion of the stem-like multipotential dedifferentiated cells thus providing the capacity for new healthy cells to grow from the stem-like cells so injected.

15. The method of claim 14 wherein following claim 14 step c) is replaced with the following additional processing comprising the steps of:
d) introducing into the large number of dedifferentiated cells, a plurality of target seed cells from a donor patient, with the target seed cells in the;
e) passing around and/or through the plurality of target seed cells an electric current stimulating the plurality of target seed cells to produce and release depressors from the seed cell membranes into the cellular cytoplasm of said seed cells which derepress genes in the seed cell nuclei allow the nuclei to manufacture new messenger RNA from the seed cells which instructs the stem-like multipotential dedifferentiated cells to redifferentiate into replicas of the target seed cells; and,
f) introducing the replicas of target seed cells into the patient donor to treat the patient donor's diseased or injured organism or the patient donor's diseased or injured collection of cells, such as tissue or blood, of the seed cell type produced.

16. The method of claim 15 wherein claim 15 step f) is replaced with the following additional processing comprising the step of:
f) growing the replicas of seed cells into a cell mass, such as an organ, which is stored and implanted into a patient for therapeutic purposes.

17-22. (canceled)

23. Isolated stem-like cells derived from fibroblasts or other blast cells, which can be made and employed to treat a donor patient for a disease or injury with a collection of cells of said stem-like cells, in the same fashion as embryonically-derived stem cells or stem-cells derived from bone marrow.

24. Isolated stem-like cells from claim 23 and related tissue suitable for culture into tissues and organs for transplant or implant into a donor patient or patient sibling, parent or child, or selected recipient.

25. Isolated stem like cells from claim 23 and related tissue suitable for culture to blood for transfusion into the donor patient or patient sibling, parent or child, or selected recipient.

26. Isolated stem-like cells from claim 23 and related tissue suitable for culture to specific types of immune system cells are concentrated and used as therapy for immune-compromised patients.

27. (canceled)

28. A method to treat a patient by implanting the isolated stem-like cells into compatible tissue of recipient at spinal cord injury sites to grow a replacement segment of cord and restore function to paralyzed areas of recipient's body.

29-34. (canceled)

35. The method of claim 13, wherein the stem-like cells involve ectodermal, entodermal or mesodermal cells from the donor patient which said ectodermal, entodermal or mesodermal cells experience pre-processing by gene-splicing.

36. The method of claim 13, wherein the target seed cell from a donor patient experience pre-processing by gene-splicing.
